(19) United States
(12) Patent Application Publication
(10) Pub. No.: US 2006/0161177 A1
(43) Pub. Date: Jul. 20, 2006

(54) Cardiac instrument for coronary sinus access through the right subclavian veins and method of using the same

(76) Inventors: Seth Worley, Lancaster, PA (US); Paul Kurth, Santa Barbara, CA (US)

Correspondence Address: Daniel L. Dawes;Myers Dawes Andras & Sherman LLP, Suite 1150, 19900 MacArthur Boulevard, Irvine, CA, 92612, US

(21) Appl. No.: 11/314,376

(22) Filed: Dec. 20, 2005

Related U.S. Application Data
(60) Provisional application No. 60/642,288, filed on Jan. 7, 2005.

(51) Int. Cl. A61B 19/00

(52) U.S. Cl. 606/129

(57) ABSTRACT

An introducer used for access to the coronary sinus from the right subclavian vein has a body and a distal tip, a hemostatic valve, a side arm and a side arm valve coupled to the hemostatic valve. The body is coupled to valve. The introducer comprises at least one straight proximal section; and at least two curved sections with opposite curvature. The straight and curved sections collectively have a length and shape to dispose the distal tip at or near the coronary sinus when accessed from the right subclavian vein. The introducer may further comprise a straight section between the two curved sections. In another set of embodiments the proximal straight and curved sections lie in a common plane. In still another set of embodiments the curved sections lie in planes which are angularly oriented with respect to each other so that the introducer is three-dimensional.

### RELATED APPLICATIONS

The present application is related to U.S. Provisional Patent Application, Ser. No. 60/642,288, filed on Jan. 7, 2005, which is incorporated herein by reference and to which priority is claimed pursuant to 35 USC 119.

### BACKGROUND OF THE INVENTION

1. Field of the Invention

The invention relates to the field of cardiac introducers which are introduced into the right atrium through the subclavian vein into cardiomyopathic hearts.

2. Description of the Prior Art

The implantation of pacing leads into the coronary sinus and the venous system of the heart have increasingly proved to be a productive approach to treatment of various cardiac electrophysiological defects and diseases. In addition, access into the coronary sinus located in the right atrium of the heart can be achieved from either a left or right superior access through the subclavian veins or a left or right inferior access through the femoral veins.

An approach to the coronary sinus and the venous system of the heart through the right subclavian vein, although not the usually preferred access route to the heart, must nevertheless be taken because the left side subclavian vein is unavailable or less inviting for any one of a number of reasons, such as infection, thrombosis, or stenosis in the left side subclavian vein or the existence of a previous right side pacer lead implant and/or the task being the implantation of an upgraded pacemaker using previously implanted leads. However, accurate or reliable access to the os of the coronary sinus of a beating heart is by no means trivial and is made even more difficult since there are acute curvatures in the right side subclavian access route and since the target heart of the present invention is a cardiomyopathic or enlarged heart which has an altered anatomy, which may vary from patient to patient depending on the disease state. The right subclavian vein joins the superior vena cava vein at an angle that can vary anywhere from 120° to 20° with the most common angulation being 60° to 70° with respect to the superior vena cava vein. The introducer must be able to navigate this subclavian-superior vena cava bend and end up being positioned in the right atrium to place its distal tip in the vicinity of or in the coronary os or coronary sinus in a myopathic heart.

The finest implantation strategy and pacing treatment in the world is worth nothing if the cardiologist cannot access the coronary sinus through the os. Access must be provided in a minimal of time with a minimal amount skill and effort in a wide variety of heart anatomies, including diseased states.

It turns out that an unbiased or a simply biased steerable introducer is ill adapted for successful coronary sinus access. It is not possible through analytic or anatomical means to predict what shape introducer is needed to provide reliable and quick coronary sinus access from any one of the approaches, including the right subclavian route. The shape and its success rate for coronary sinus access is determined according to the invention to be dependent not only on the internal cardiac anatomy, but also on the shape of the vascular path from the percutaneous insertion site to the right atrium and coronary os.

Therefore, what is needed is some kind of definition of an introducer shape or shapes, which systematically yield reliable, successful and easy-to-use introducers for coronary sinus access through the right subclavian vein.

### BRIEF SUMMARY OF THE INVENTION

The illustrated embodiment of the invention is an introducer having a distal tip and used for access to the coronary sinus from the right subclavian vein. The introducer has a body and further comprising a hemostatic valve; and side arm and side arm valve coupled to the hemostatic valve, where the body is coupled to valve. The introducer comprises at least one straight proximal section; and at least two curved sections with opposite curvature. The straight and curved sections collectively have a length and shape to dispose the distal tip at or near the coronary sinus when accessed from the right subclavian vein.

The introducer may further comprise a straight section between the two curved sections.

In one set of embodiments each curved section is connected to the next curved section by a single straight section of the body.

In another set of embodiments the proximal straight and curved sections lie in a common plane.

In still another set of embodiments the curved sections lie in planes which are angularly oriented with respect to each other so that the introducer is three-dimensional.

For example, the introducer has a body which comprises a proximal straight section, a first curved section R1 having a curvature with a defined sense of curvature and contiguous with the proximal straight section, a first straight section S1 contiguous with the first curved section R1, a second curved section R2 having a curvature opposite to the defined sense of curvature and contiguous with the first straight section S1, a second straight section S2 contiguous with the second curved section R2, and a third curved section R3 having a curvature opposite to the defined sense of curvature and contiguous with the second straight section S2.

The proximal straight section is has a length in the range of 1.00-5.00 inches and is followed by the first curved section R1 which has a radius of curvature in the range of approximately 1.72-2.34 inches. The angle included between the proximal straight section and the distal end of the first straight section S1 is in the range of approximately 70°-130°. The first straight section S1 has a length approximately in the range of 0-3.5 inches. The second curved section R2 may have a single radius of curvature or be comprised of a plurality of curved sections, each which are oppositely directed to the radius of the curvature of the first curved section R1 in the planar configuration of body.

Alternatively described it can be said that the introducer has a body which is comprised of a plurality of sections of prebiased straight sections alternating with a plurality of prebiased curved sections including the two curved sections of opposite curvature. All sections lie in a common plane or each or selected ones of the curved sections and/or each or selected ones of the straight sections extend above or below a common plane by selected angular degrees. In the last case the curved and straight sections lie in a plurality of planes, each of the planes being inclined with respect to each preceding one of the planes by an increasing dihedral angle relative to a first one of the planes.

For example in the three dimensionally biased introducer the plurality of planes comprise four planes, the first of which is designated as the horizontal plane and the three succeeding planes being inclined with respect to the horizontal plane at 10°±10°, 30°±2° and 55°±20° respectively. A second plane of the four planes is adjacent the horizontal plane and one curved and one straight section lie in the second plane, where a third plane of the four planes is adjacent the second plane and one curved and one straight section lie in the third plane, where a fourth plane of the four planes is adjacent the third plane and one curved section lies in the fourth plane.

In yet another characterization of the invention, it can be described as an introducer for access to the coronary sinus from the right subclavian vein having a plurality of biased sections comprising a proximal straight section, at least two curved sections of opposite curvature, and a distal end.

The straight and curved sections lie in a common plane or be inclined out of the plane in which the proximal section lies.

The distal end may comprise a distal section with straight or curved shape.

The introducer may further comprise a straight section disposed between the two curved sections.

In the simplest embodiment the two curved sections comprise a proximal curved section and a distal curved section. The distal curved section collectively comprises a plurality of curved or straight sections.

In another embodiment the distal curved section collectively comprises three curved sections, the distal most one of the three curved sections being the distal end.

The distal curved section may also collectively comprise one curved section and one straight section, the distal most one being the straight section, which is the distal end of the introducer.

The distal most straight section is generally parallel with the proximal straight section or inclined with respect to the proximal straight section at an angle between 75° and 200°. For example, the distal most straight section is generally inclined with respect to the proximal straight section at an angle approximately 114°. Alternatively in the simplest embodiment having only the proximal straight section and two curved sections, the proximal straight section is generally inclined with respect to the extended longitudinal axis of the distal end at an angle approximately 116°.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side plan view of an introducer with an in-plane curvature according to the invention.

FIG. 2 is a dimensioned side plan view of an introducer of the embodiment shown in FIG. 1.

FIG. 2a is a side plan view of an introducer of the embodiment shown in FIGS. 1 and 2 more graphically depicted as an alternating series of straight and curved sections or sections.

FIG. 3 is a perspective view of another class of embodiments of the invention wherein the introducer's sections lie out of plane with respect to each other.

FIG. 4 is a side plan view of an introducer of another embodiment lying in a common plane.

FIG. 5 is a side plan view of an introducer of still another embodiment lying in a common plane.

FIG. 6 is a side plan view of an introducer of yet another embodiment lying in a common plane.

FIG. 7 is a side plan view of an introducer of a basic embodiment lying in a common plane.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

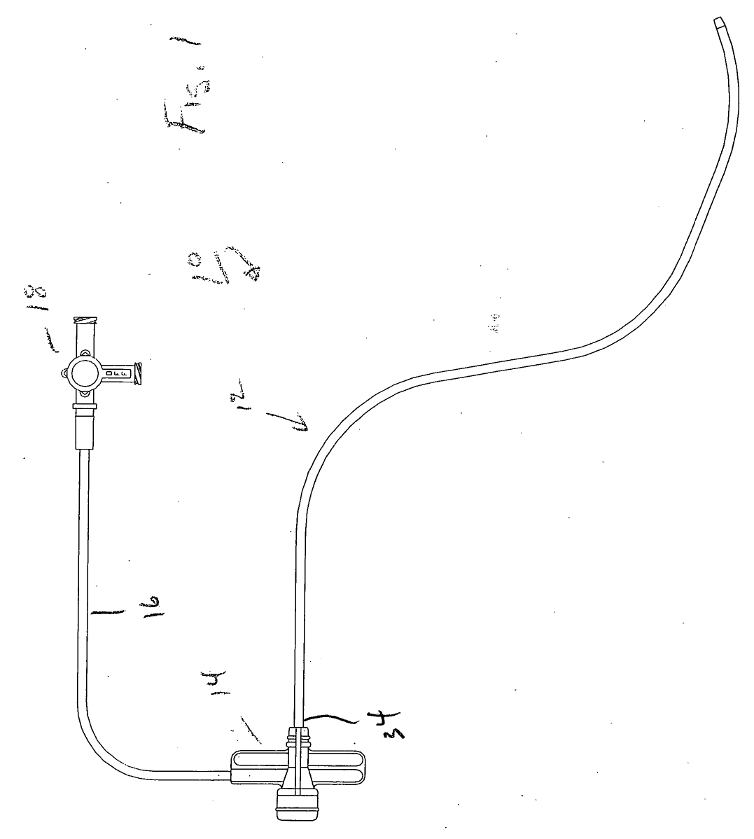

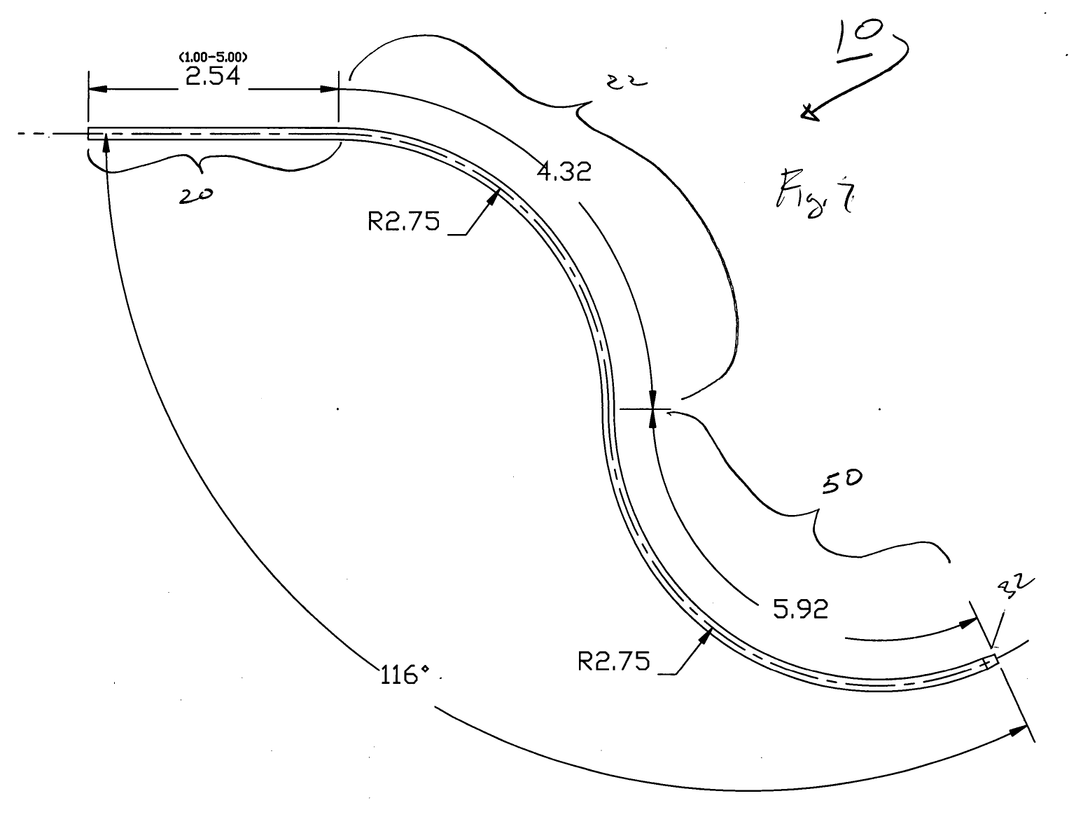

Before considering the more complex shapes of the embodiments of FIGS. 1-6, consider first the basic embodiment shown in FIG. 7 of introducer 10, wherein only the body of the introducer is depicted. The embodiment of FIG. 7 is the simplest shape illustrated and is comprised of a straight proximal section 20 approximately 2.54 inches in length, but which should be considered as having any length within the range of approximately 1-5 inches. Straight section 20 is contiguous with a prebiased first curved section 22 approximately 4.32 inches in length, but which should be considered as having any length within the range of approximately 1.5-4.5 inches. The radius of curvature of first curved section 22 shown in FIG. 7 is approximately 2.75 inches and lies on the left side of introducer 10 as viewed in FIG. 7. This could be defined for purposes of illustration as having an inward or negative curvature.

First curved section 22 is contiguous with a prebiased second curved section 50 which is approximately 5.92 inches in length, but which should be considered as having any length within the range of approximately 2 to 10 inches. The radius of curvature of second curved section 50 shown in FIG. 7 is approximately 2.75 inches and lies on the right side of introducer 10 as viewed in FIG. 7. This could be defined for purposes of illustration as having an outward or positive curvature.

In its simplest embodiment, introducer 10 is thus characterized by a proximal straight portion 20, a first curved section 22 with a first sense of curvature and a second curved section 50 with a second sense of curvature opposite to the first sense of curvature. The various sections of introducer 10 are sized and shaped so that distal tip 32 of introducer 10 is situated at or near the coronary os in a cardiomyopathic heart when implanted through the right subclavian vein. The included angle between proximal section 20 and the extended longitudinal axis of introducer 10 at distal tip 32 is approximately 116°, but may take any value between 75°-200°.

The introducer 10 is shown in side plan view in FIG. 1 and is comprised of an introducer body, generally denoted by reference numeral 12, separable hemostatic valve 14, side arm 16 and a controllable side arm T-valve 18. The introducer 10 is used for pacemaker lead implantations or for the insertion or implantation of any other instrument or object into the heart. Separable hemostatic valve 14, side arm 16 and a side arm T-valve 18 are each conventional and may assume any form, shape, design or structure now known or later devised without alteration of the scope of the invention. The diameter of introducer 10 may be chosen according to design and the invention is independent of the details of the interior structure of the introducer, which may incorporate any design or structure now known or later devised.

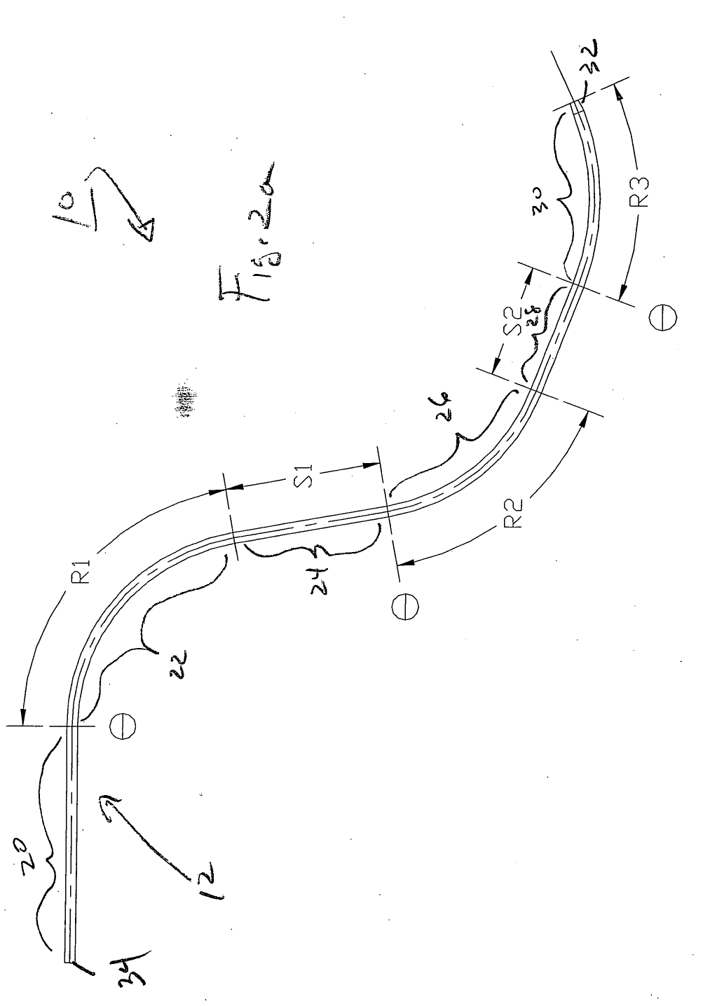

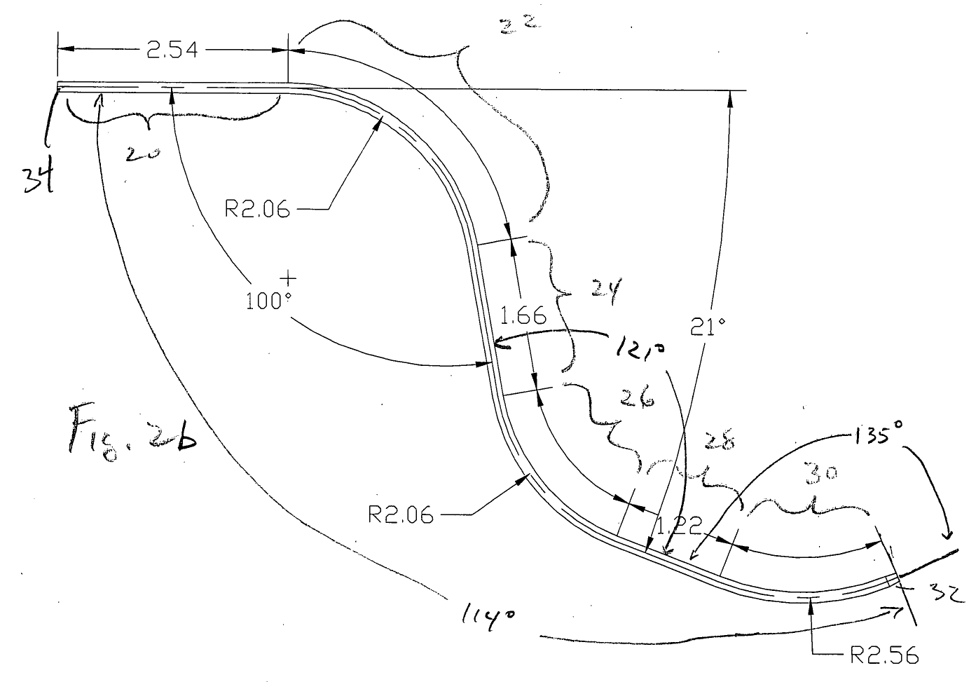

Body 12 of introducer 10 is prebiased by conventional means including any means now known or later devised to assume the shape as shown in plan view in FIGS. 1 and 2 and as further disclosed here, which shape is particularly adapted to allow entry into the coronary sinus of the heart through subcutaneous access through the right side subclavian vein. Therefore, when a section of the introducer is characterized as being straight or curved, it is to be understood that the described shape is what the section assumes in the absence of external forces acting on the section and not that the section is rigid or incapable of assuming a different shape. The introducer 10 and all of its sections are pliable and are capable of conforming to the shapes of the human vascular system. However, when left to their own devices, the sections will tend to return or to remember their biased shape.

Body 12 of introducer 10 is considered to have a general or arbitrary structure in all design aspects other than as may be specifically limited in the claims and may, for example, be separable by any means now known or later devised, reinforced or nonreinforced. The embodiment of FIG. 1 is shown to lie entirely within a single plane as described below, but it is to be understood that any of the delineated sections may also be biased to lie out of plane.

In the illustrated embodiment as best shown in the plan view of FIG. 2, body 12 is comprised of alternating sections of prebiased straight sections 20, 24, and 28 and prebiased curved sections 22, 26, and 30. The curved sections 22, 26, and 30 may be curved in the same or in differing directions, i.e. the center of curvature may lie on the same or different sides of introducer 10 as seen in FIGS. 1 and 2. In the illustrated embodiment all sections 20-30 lie in the plane of the figure, but it is also expressly understood that in each or selected ones of the curved sections 22, 26, and 30, and/or in each or selected ones of the straight sections 20, 24, and 28 body 12 may be biased to extend above or below the plane of FIGS. 1 and 2 by selected angular degrees or ranges of degree.

From the viewpoint of FIGS. 1 and 2, without quantification of the lengths and the radii of curvature, starting from the proximal end 34 coupled to valve 14 and moving to the distal end 32, introducer 10 is comprised in sequence of a straight section 20, a clockwise curving section 22, a straight section 24, a counterclockwise curving section 26, a straight section 28 and a counterclockwise curving section 30. Out of plane orientations are possible for any of the sections, but preferably for the curved sections 24, 26 and 30.

As generally shown in FIG. 2a introducer 10 is comprised a first straight section 20 which in the illustrated embodiment is approximately 2.54 inches (but in the range of 1-5 inches) and is followed by first curved section 22 having a length of approximately 2.87 inches and a radius of curvature of approximately 2.06 inch (but in the range of 1.72-2.34 inches). The length of curved section 22 is such that the included angle between adjacent straight sections 20 and 24 is approximately 100° (but within the range of 70°-130°)

First curved section 22 is followed by a second straight section 24, which has a length of approximately 1.66 inch (but in the range of 0-3.5 inches). Second curved section 26 is contiguous with a second straight section 24 and has a radius of curvature which is oppositely directed to the radius of curvature of first curved section 22 in the planar configuration of body 12. Second curved section 26 has a radius of curvature of approximately 2.06 inch (but in the range of 1.9-2.5 inches) and a length of approximately 2.5, but in the range of approximately 1-4 inches so that the included angle between the extension of straight section 20 and straight section 28 is approximately 21° but in the range of 15° to 30°.

A third straight section 28 having a length of approximately 1.22 inch, but in the range of 0.5 to 2.5 inches follows second curved section 26. Finally, body 12 terminates with a distal third curved section 30 which has a radius of curvature, which is oppositely directed to the radius of curvature of first section 22 and is curved in the same direction as second curved section 26 in the planar configuration of body 12. The radius of curvature of third curved section 30 is approximately 2.56 inch but in the range of 0.5 to 3.0 inches. Section 30 preferably has a length approximately 2.02 inches but is in the range of 0.5 to 5.0 inches The distal tip 32 may be provided with a radioopaque marker and may be tapered or rounded.

Preferred values for numerical illustrations are given, but it must be understood that the parameter in question may assume any value within the recited range associate with it. It must also be understood that the above ranges are not tolerances of the various sections of introducer 10, but are the ranges which are contemplated as included within the various embodiments. The length of each section may be independently varied by plus or minus 30% without departing from the scope of the invention. The ranges may be in any given embodiment be more limited than that listed above in order to provide the desire right atrial access into the heart according to the geometry of the heart in question, which will vary not only based on individual anatomical variation, and body size, but also on the nature and the degree of any cardiac disease state that might exist.

FIG. 2a illustrates the geometric elements of the body 12 of introducer 10, and provides another viewpoint of the various embodiments of the invention, namely curved sections R1, R2 and R3 alternated by straight sections S1 and S2. The sense of the curvature of R1 being opposite that of R2 and R3. In the illustrated embodiment of FIG. 2, the included angle between the straight or proximal section 20 and S1, straight section 24, is approximately 100°±30°. The included angle between S1, the straight section 24, and S2, straight section 26, is approximately 121°±36°. The included angle between S2, the straight section 28, and the extension of the axis at distal tip 34 of R3, curved section 30, is approximately 135°±41°. The angle between an extension of the longitudinal axis of proximal straight section 20 and a perpendicular to the axis at distal tip 34 of R3, the curved section 30, is approximately 114°±34°. The angles specified determine the relative lengths or proportions of the sections S1, S2, R1, R2 and R3. The overall size of introducer 10 can then be scaled up or down with the relative proportions held within their specified ranges, even though the lengths may not be within the quantitative ranges first disclosed above.

The distal end of body 12 may be provided with a radioopaque marker 32 or a tapered and/or lubricated tip 32 to facilitate insertion into the coronary sinus or within a telescopic introducer system. It must be understood that introducer 10 may be employed without combination with other instruments, or may be employed with another instrument such a dilator or telescopic introducer. The dilator is telescopically disposed in a lumen (not shown) defined in introducer 10 to tend to straighten introducer 10 while it is being inserted into the vascular system. Introducer 10 may also be telescopically disposed through a lumen in a telescopic second introducer (not shown) which modifies the curvature of introducer 10 to assist in its placement at a predetermined position with in the vascular system or more particularly, the coronary sinus.

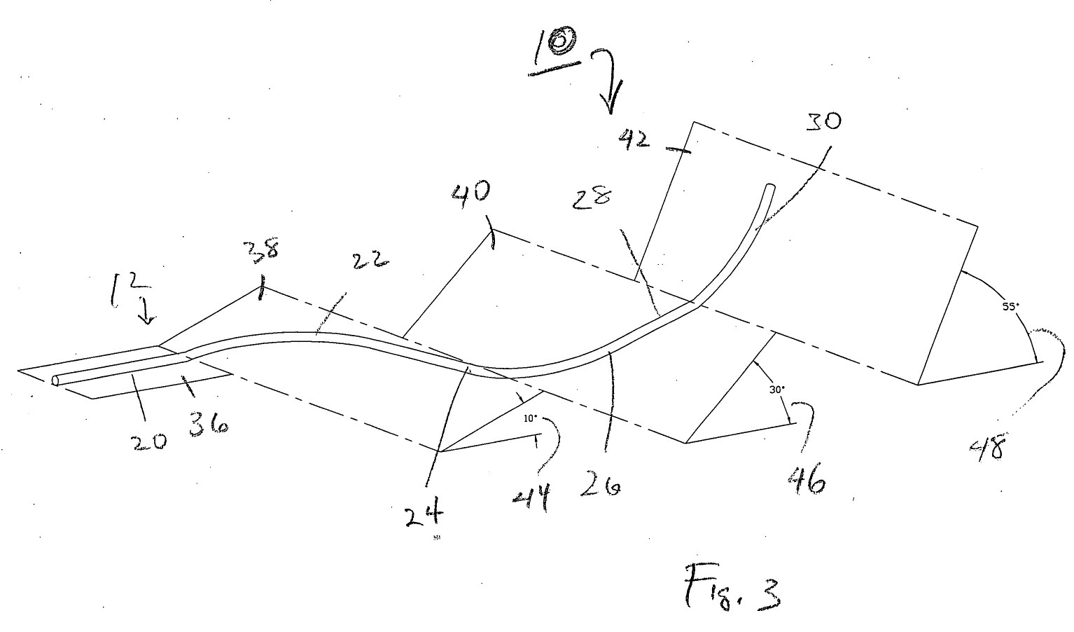

FIG. 3 is a three dimensional depiction of body 12 of introducer 10 shaped in the illustrated embodiment in three dimensions in four dihedral planes 36-42. Straight section 20 is disposed in a plane 36, which may be referenced as the horizontal plane, although designation of which plane is horizontal is arbitrary. Inclined at a first dihedral angle 44 relative to plane 36, which angle 44 is taken to be approximately 10°±10° is a second plane 38. Curved section 22 and straight section 24 lie in the plane 38, although straight section 24 may be arranged to lie in the third dihedral plane 40 if desired. Inclined with respect to plane 38 at a second dihedral angle 46 is the third plane 40, which angle 46 is taken to be approximately 30°±20° relative to the horizontal. Curved section 26 and straight section 28 lie in the plane 40, although straight section 28 may be arranged to lie in a fourth dihedral plane 42 if desired. Inclined at a third dihedral angle 48 to plane 40 is the fourth plane 42, which angle 48 is taken to be approximately 55°±20° relative to the horizontal. Curved section 30 lies in the plane 42. The dihedral angles 44-48 may be altered according to the desired cardiac access intended for introducer 10, which is a function of the geometry of the cardiovascular system.

Further, in addition to specifying dihedral angles between planes 36-42, their relative angular orientation may be entirely general so that three angles may need to be specified to define the relative angular orientation of each plane to the adjacent planes instead of a single dihedral angle, giving a total of nine relative angles needed to fully define the three dimensional shape of introducer 10. Again, the three, three-dimensionally angularly oriented planes may be oriented according to the desired cardiac access intended for introducer 10, which is a function of the geometry of the cardiovascular system.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments. For example, it is to be understood that if the illustrated embodiment of FIG. 2 were reflected about the longitudinal axis of section 20 that the sense of curvature would then be oppositely described, i.e. curved section 22 would curve in a counterclockwise direction instead of a clockwise direction and the sense of curvature for curved sections 26 and 30 would similarly be reversed. The topology that curved section 22 has an opposite sense of direction of curvature from curved sections 26 and 30 remains the same. Furthermore, the radii of curvature of curved sections 26, 28 and 30 have been described by a single number with the assumption that the curved sections 26, 28 and 30 are simple circular arcs. However, curved sections 26, 28 and 30 may be compound arcs as well in which the radius of curvature assumes a different value as each point on the curved sections 26, 28 and 30. The radius of curvature shown then in FIG. 2 and described above may then be considered to be the average radius of curvature for the curved section 26, 28 and 30. Still further, the magnitude of the various lengths and curves in the illustrated embodiment must be expressly understood to be an example only and not to limit the more general topology of the introducer body 12.

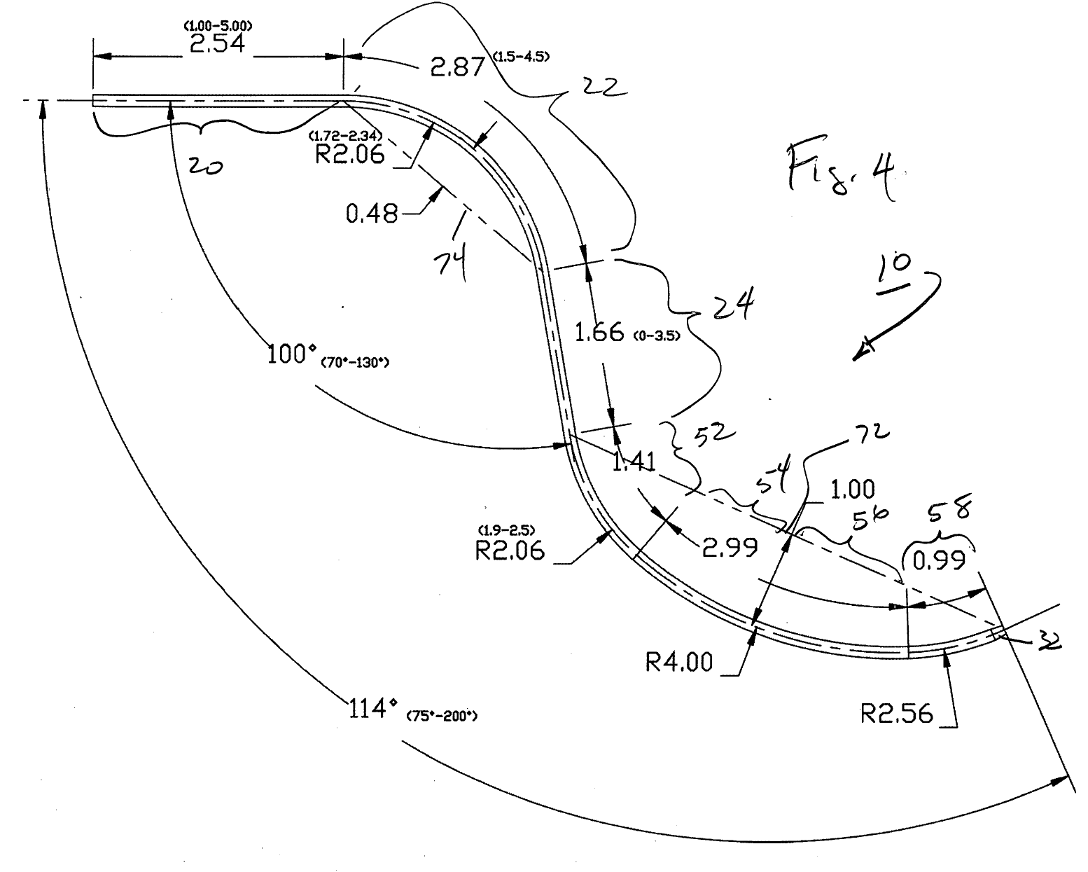

FIG. 4 is a side plan view of another embodiment lying in a common plane. Here proximal section 20 is contiguous with first curved section 22 which preferably has a length of approximately 2.87 inches, but again may take any value in the range of 1.5-4.5 inches. The radius of curvature of section 22 in FIG. 4 is approximately 2.06 inches and is inward. This results in a chord 74 for section 22 with a height of approximately 0.48 inch, but in the range of 0.2 to 1.5 inch. However, the radius of curvature of section 22 may take on any value between 1.72 and 2.34 inches.

First curved section 22 is then followed by straight section 24 which preferably has a length of approximately 1.66 inches, but again may take any value in the range of approximately 0-3.5 inches. The included angle between proximal section 20 and straight section 24 is approximately 100°, but may take any value between 70°-130°.

Straight section 24 is contiguous with a compound curved section comprises of separated curved sections 52-58. Curved section 52 has an outward radius of curvature of approximately 2.06 inches, but may take on any value in the range of approximately 1.9-2.5 inches. Curved section 52 has a length of approximately 1.41 inches but in the range of 0.5-3.0. The next contiguous curved section 54 has a length of approximately 2.99 inches and has a radius of outward curvature of approximately 4.00 inches, but may take on any value in the range of approximately 2.0-5.0 inches. Finally the distal most section 58 is also outwardly curved radius of approximately 2.56 inches, but may take on any value in the range of approximately 2 to 3 inches. Distal most section 58 has a length of approximately 0.99 inch but in the range 0-2.0. The compound curve comprised of sections 52-58 has a collective span 72 with a height above the curve of approximately 1.00 inch but in the range of 0.2-3.0 inches. The included angle between proximal section 20 and the extended longitudinal axis of introducer 10 at distal tip 32 is approximately 114°, but may take any value between 75°-200°.

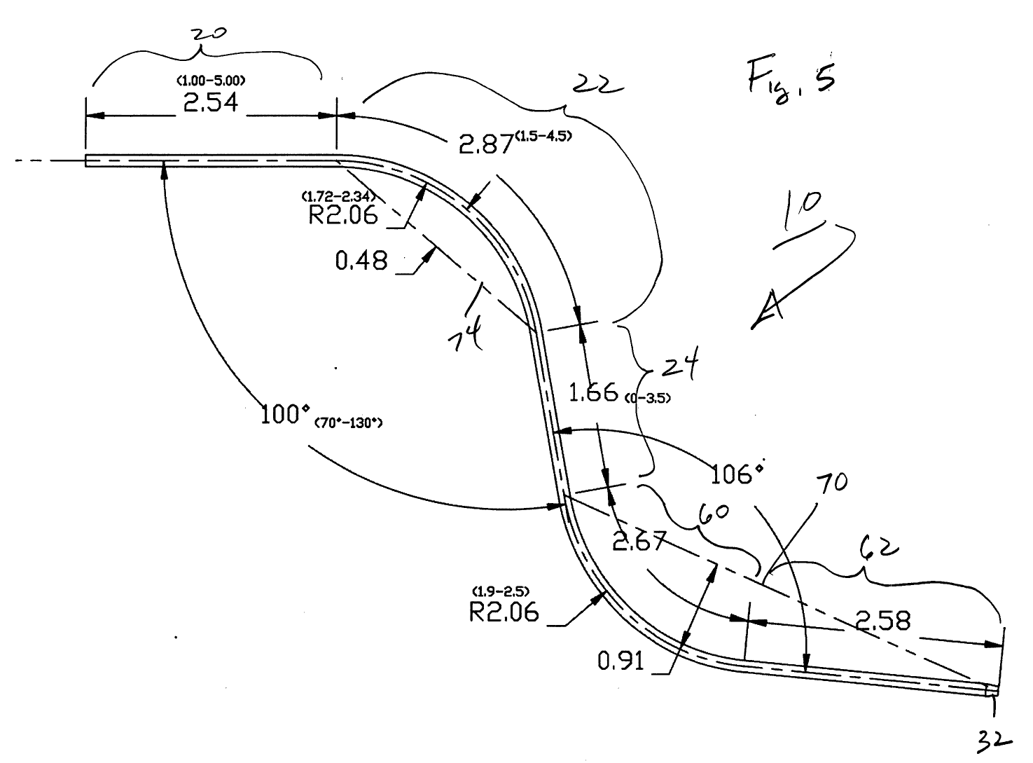

Yet another embodiment is shown in FIG. 5, which is a side plan view of another embodiment lying in a common plane. Sections 20-24 of FIG. 5 are identical to that described in FIG. 4 and will therefore not be redescribed here. Straight section 24 is contiguous with a single curved section 60 having an approximate length of 2.67 inches, but may take on any value in the range of approximately 1.5 to 4.0 inches. Curved section 60 again has an outward radius of curvature of approximately 2.06 inches, but may take on any value in the range of approximately 1.9-2.5 inches. The curvature of section 60 is thus identical to that described in connection with section 52 of FIG. 4, but is longer in length. Finally the distal most section 62 is straight with a length of approximately 2.58 inches, but may take on any value in the range of approximately 0.5-4.0 inches. The span 70 from the proximal end of section 60 to the distal end of section 62 has a height of approximately 0.91 inch above the curve, but may take on any value in the range of approximately 0.2-3.0 inches.

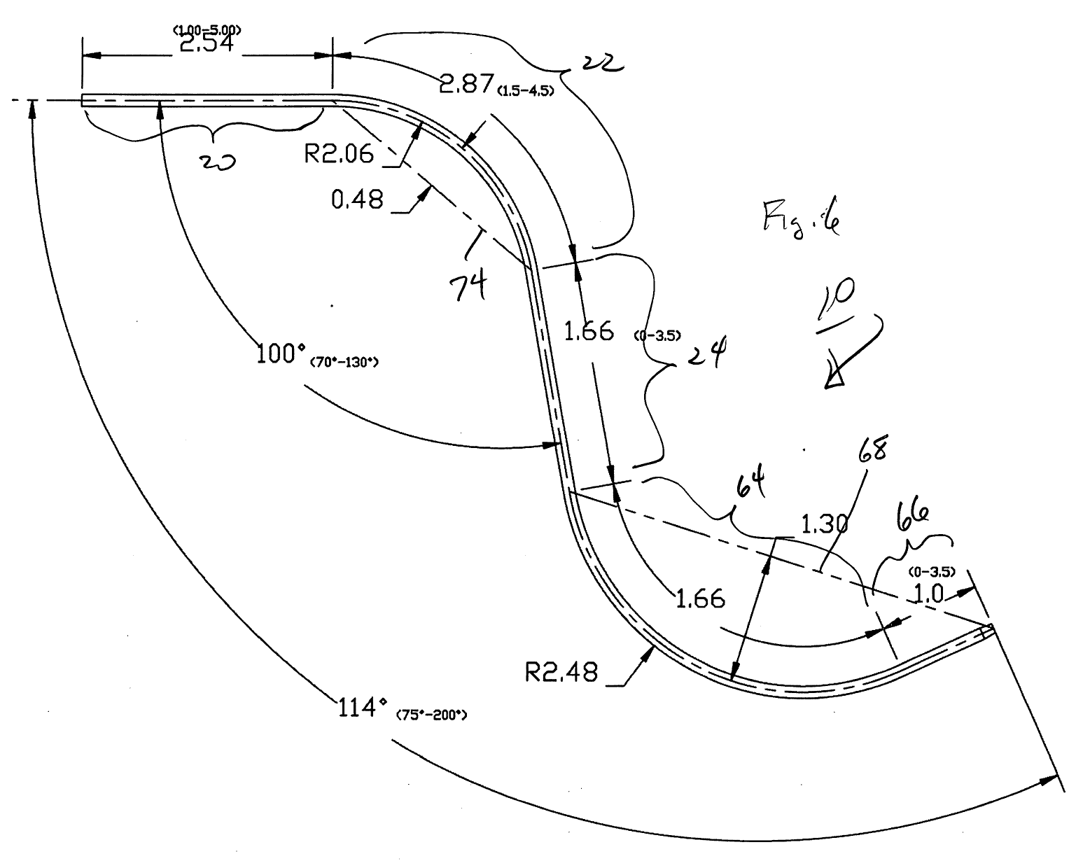

The embodiment of FIG. 6 is topologically similar to that shown in FIG. 5, but is differently dimensioned and shaped. FIG. 6 is a side plan view of still another embodiment lying in a common plane. Sections 20-24 of FIG. 6 are identical to that described in FIG. 4 and will therefore not be redescribed here. Straight section 24 is contiguous with a single curved section 64 having an approximate length of 1.66 inches, but may take on any value in the range of approximately 0.5 to 3.0 inches. Curved section 64 has an outward radius of curvature of approximately 2.48 inches, but may take on any value in the range of approximately 1.9-2.5 inches. Finally the distal most section 66 is straight with a length of approximately 1.0 inches, but may take on any value in the range of approximately 0-3.5 inches. The span 68 from the proximal end of section 60 to the distal end of section 62 has a height above the curve of approximately 1.30 inch, but may take on any value in the range of approximately 0.5-3.0 inches.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

For example, wherever a curved section has been described, it has been characterized by a circular curvature characterized at each point by a single radius of curvature. However, it is to be understood that the radius of curvature may also vary from point to point on the curved sections and only be approximated by the described single radius of curvature. The object is to place distal tip 32 at, near or in the coronary os or into the coronary sinus in myopathic hearts when accessing the heart through the right side subclavian vein. Different ones of the embodiments can be advantageously employed in different myopathic hearts taking on different anatomies based on their diseased states. Each of the described dimensions of curvature and length can thus be independently altered or variously scaled to achieve this same object according to the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An introducer having a distal tip and used for access to the coronary sinus from the right subclavian vein comprising:
at least one straight proximal section; and
at least two curved sections with opposite curvature, the straight and curved sections collectively having a length and shape to dispose the distal tip at or near the coronary sinus when accessed from the right subclavian vein.

2. The introducer of claim 1 further comprising a straight section between the two curved sections.

3. The introducer of claim 2 where each curved section is connected to the next curved section by a single straight section of the body.

4. The introducer of claim 1 where the proximal straight and curved sections lie in a common plane.

5. The introducer of claim 2 where the straight and curved sections lie in a common plane.

6. The introducer of claim 1 where the curved sections lie in planes which are angularly oriented with respect to each other so that the introducer is three-dimensional.

7. The introducer of claim 2 where the curved sections lie in planes which are angularly oriented with respect to each other so that the introducer is three-dimensional.

8. The introducer of claim 2 where the body comprises a proximal straight section, a first curved section R1 having a curvature with a defined sense of curvature and contiguous with the proximal straight section, a first straight section S1 contiguous with the first curved section R1, a second curved section R2 having a curvature opposite to the defined sense of curvature and contiguous with the first straight section S1, a second straight section S2 contiguous with the second curved section R2, and a third curved section R3 having a curvature opposite to the defined sense of curvature and contiguous with the second straight section S2.

9. The introducer of claim 2 where the body is comprised of a plurality of sections of prebiased straight sections alternating with a plurality of prebiased curved sections including the two curved sections of opposite curvature.

10. The introducer of claim 9 where all sections lie in a common plane.

11. The introducer of claim 9 where each or selected ones of the curved sections and/or each or selected ones of the straight sections extend above or below a common plane by selected angular degrees.

12. The introducer of claim 11 where the curved and straight sections lie in a plurality of planes, each of the planes being inclined with respect to each preceding one of the planes by an increasing dihedral angle relative to a first one of the planes.

13. The introducer of claim 12 where the plurality of planes comprise four planes, the first of which is designated as the horizontal plane and the three succeeding planes being inclined with respect to the horizontal plane at 10°±10°, 30°±20° and 55°±20° respectively.

14. The introducer of claim 13 where a second plane of the four planes is adjacent the horizontal plane and one curved and one straight section lie in the second plane, where a third plane of the four planes is adjacent the second plane and one curved and one straight section lie in the third plane, where a fourth plane of the four planes is adjacent the third plane and one curved section lies in the fourth plane.

15. The introducer of claim 11 where the proximal straight section is approximately 2.54 inches long but lies within the range of 1.0 to 5.0 inches, and is followed by the first curved section R1 which has a radius of curvature of approximately 2.06 inches but in the range of 1.72-2.34 inches and an angular segment of approximately 100° but within the range of 70°-130°, followed by the first straight section S1 which is approximately 1.66 inches long but in the range of 0-3.5 inches, a second curved section R2 with a radius of curvature of approximately 2.06 inches but in the range of 1.9-2.5 inches which is oppositely directed to the radius of the curvature of the first curved section R1 in the planar configuration of body, where a longitudinal axis of the second straight section S2 or the extended distal direction of the second curved section R2 defines an angle of approximately 21° with the longitudinal axis of the proximal straight section but in the range of 15° to 30°, where the second straight section S2 is approximately 1.22 inches long but in the range of 0.5 to 2.5 inches, and a distal third curved section R3 which has a radius of curvature of approximately 2.56 inches but in the range of 0.5 to 3.0 inches, and a length approximately 2.02 inch but in the range of 0.5 to 5.0 inches, which is oppositely directed to the radius of curvature of first curved section R1 and is curved in the same direction as second curved section R2 in the planar configuration of the body.

16. The introducer of claim 1 where the introducer has a body and further comprising a hemostatic valve; and side arm and side arm valve coupled to the hemostatic valve, where the body is coupled to valve.

17. An introducer for access to the coronary sinus from the right subclavian vein having a plurality of biased sections comprising:
a proximal straight section;
at least two curved sections of opposite curvature; and
a distal end.

18. The introducer of claim 17 where the straight and curved sections lie in a common plane.

19. The introducer of claim 17 where the straight and curved sections lie in at least two different planes.

20. The introducer of claim 17 where the distal end comprises a distal section with straight or curved shape.

21. The introducer of claim 17 further comprising a straight section disposed between the two curved sections.

22. The introducer of claim 17 where the two curved sections comprise a proximal curved section and a distal curved section, the distal curved section collectively comprises a plurality of curved or straight sections.

23. The introducer of claim 22 where the distal curved section collectively comprises three curved sections, the distal most one of the three curved sections being the distal end.

24. The introducer of claim 22 where the distal curved section collectively comprises one curved section and one straight section, the distal most one being the straight section, which is the distal end of the introducer.

25. The introducer of claim 24 where the distal most straight section is generally parallel with the proximal straight section.

26. The introducer of claim 24 where the distal most straight section is generally inclined with respect to the proximal straight section at an angle between 75° and 200°.

27. The introducer of claim 26 where the distal most straight section is generally inclined with respect to the proximal straight section at an angle approximately 114°.

27. The introducer of claim 17 where the proximal straight section is generally inclined with respect to the extended longitudinal axis of the distal end at an angle approximately 116°.
